(19) United States
(12) Patent Application Publication
(10) Pub. No.: US 2023/0061636 A1
(43) Pub. Date: Mar. 2, 2023

(54) ASSESSING A PATIENT STATE

(71) Applicant: BIOGEN MA INC., Cambridge, MA (US)

(72) Inventors: Peter BERGETHON, Dover, MA (US); Sheraz KHAN, Quincy, MA (US); Krishna KILAMBI, Malden, MA (US); Jason OSIK, Wakefield, MA (US)

(73) Assignee: BIOGEN MA INC., Cambridge, MA (US)

(21) Appl. No.: 17/904,789

(22) PCT Filed: Feb. 23, 2021
(86) PCT No.: PCT/US2021/019174

§ 371 Date: Aug. 23, 2022

Related U.S. Application Data
(60) Provisional application No. 62/980,528, filed on Feb. 24, 2020.

(51) Int. Cl. G16H 50/50; G16H 10/60; G16H 40/67; G16H 50/30

(52) U.S. Cl. CPC G16H 50/50; G16H 10/60; G16H 40/67; G16H 50/30

(57) ABSTRACT

A medical process comprising: identifying one or more symptoms, determining at least one measurement descriptive of the one or more symptoms, creating a model of the at least one measurement, obtaining the at least one measurement descriptive of the one or more symptoms from at least one symptomless subject, using the model to transform the at least one measurement from the at least one symptomless subject into a reference data, obtaining the at least one measurement descriptive of the one or more symptoms from at least one subject with the one or more symptoms, using the model to transform the at least one measurement from the at least one subject with the one or more symptoms into a patient data, and comparing the reference data to the patient data.

### CROSS-REFERENCE TO RELATED APPLICATIONS

This application claims benefit of priority from U.S. Provisional Application No. 62/980,528, filed on Feb. 24, 2020, which is incorporated herein by reference in its entirety.

### TECHNICAL FIELD

This disclosure relates generally to systems and methods of assessing patients' states. More particularly, at least some embodiments of the disclosure relate to systems and methods of quantitatively assessing patient symptoms and comparing such assessment to a reference, e.g., reference data.

### BACKGROUND

Measuring the effect of a novel therapeutic candidate on the progression of a slow and heterogeneous disease, such as Lewy-body dementia or idiopathic Parkinson's disease (PD), is challenging. Disease severity is currently evaluated based on subjective clinical rating scales, such as the Movement Disorder Society's Unified Parkinson's Disease Rating Scale (MDS-UPDRS). Such scales typically rely on individual neurologists or physicians to estimate the severity of a patient's symptoms. Scores are assigned based on the physician's observations and past experience. As a result, subjective clinical rating scales like MDS-UPDRS may have poor sensitivity with respect to detecting small changes in a patient's state, especially in the case of slow and heterogeneous diseases like PD.

### SUMMARY OF THE DISCLOSURE

According to an example, a medical process may comprise identifying one or more symptoms, determining at least one measurement descriptive of the one or more symptoms, creating a model of the at least one measurement, obtaining the at least one measurement descriptive of the one or more symptoms from at least one symptomless subject, using the model to transform the at least one measurement from the at least one symptomless subject into a reference data, obtaining the at least one measurement descriptive of the one or more symptoms from at least one subject with the one or more symptoms, using the model to transform the at least one measurement from the at least one subject with the one or more symptoms into a patient data, and comparing the reference data to the patient data.

In another example, the at least one measurement may include cognitive or physical movement related metrics. Creating the model of the at least one measurement may include creating a mathematical model. The medical process may further comprise converting the at least one measurement from the at least one symptomless subject into a reference score, and converting the at least one measurement from the at least one subject with the one or more symptoms into a patient score. The medical process may further comprise mapping the reference data to create a reference graph, and mapping the patient data to create a patient graph. The at least one measurement descriptive of the one or more symptoms from at least one subject with the one or more symptoms may be obtained in time intervals.

In another example, the one or more symptoms may be attributed to a disease including Parkinson's Disease. The one or more symptoms may include bradykinesia, gait, and/or tremor. The at least one measurement may include movement related metrics including displacement, velocity, and acceleration of movements. Obtaining the at least one measurement descriptive of the one or more symptoms from the at least one subject with the one or more symptoms may include monitoring the at least one subject with the one or more symptoms while completing at least one task. The at least one task may include movement-related tasks. The at least one task may include wrist rotation, leg lifts, toe taps, walking, and/or postural sway. The at least one measurement may be obtained via a subject monitoring system. The subject monitoring system may be configured to monitor a motion of the at least one symptomless subject and the at least one subject with the one or more symptoms. The subject monitoring system may include at least one sensor configured to obtain the at least one measurement.

It may be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate exemplary embodiments of the present disclosure and together with the description, serve to explain the principles of the disclosure.

FIGS. 1A-1F are various embodiments of a system/device configured to capture the motion of a subject.

FIGS. 2A-C are charts illustrating the progression of PD characteristics.

FIGS. 3A-3F are charts comparing quantitative and qualitative assessments of PD characteristics.

FIGS. 4A-4D are charts illustrating the relationship between various movement metrics measured via a sensor worn by a subject.

FIGS. 5A-5F are charts comparing various movement metrics measured via a sensor worn by a subject.

FIGS. 6A-6B are diagrams illustrating exemplary processes for preparing metric calculations.

### DETAILED DESCRIPTION

This disclosure is drawn to systems and methods for assessing patients' disease states and characteristics, among other aspects. Reference will now be made in detail to aspects of the disclosure, examples of which are shown in the accompanying figures and further discussed below. Wherever possible, the same or similar reference numbers will be used through the drawings to refer to the same or like parts.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section, Both the foregoing general description and the following detailed description are exemplary and explanatory only and are not restrictive of the features, as claimed.

As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. Additionally, the term “exemplary” s used herein is used in the sense of “example,” rather than “ideal.”

Embodiments of the disclosure may save one or more of the limitations in the art. The scope of the disclosure, however, is defined by the attached claims and not the ability to solve a specific problem.

Modeling Pittman Movements

The following discussion provides background relating to human motor control. The motor manifestations seen in all natural movements—as well as in disease states—are kinematic and kinetic actions that are the emergent properties of:
a series of inter-coordinated planning and control systems (neural circuits) that plan, detect, modulate, and error-correct actions;
power generating components (muscles and neuromuscular activations) that enable physical or mechanical action; and
mechanical elements (skeletal, connective tissues) that apply these generated forces to a truss system of levers, wheels/axles, and inclined planes to cause kinematic motions.

The principal neural systems for output control include corticospinal, extrapyramidal, cerebellar, rubrospinal, and tectospinal. The principal neural systems for input control include proprioceptive, spinocerebellar, nocioceptive, and spinothalamic. The principal systems for power generation are:
ventral grey matter (anterior horn pyramidal cells);
motor neurons including the neuromuscular junction
striated muscle cells, fibers and fibrils composing muscles arranged as agonist/antagonist pairs acting to force and damp the mechanical machines that characterize the endo and exoskeleton;
rigid skeletal components that act to transduce muscular forces into actions of body component parts; and
connective tissue force transducers that attach the force generating elements to the mechanical truss that causes physical action to occur and also provides varying elements of hardness, compliance and damping parameters to the forcing functions that characterize the delivery of agonist/antagonist forces delivered as torque is applied to the skeletal truss for stability and motion.

When a movement occurs, it is the result of motor planning within “EPA” space defined here as:
Egocentric (E) space representation of the actual body surface;
Pericentric (P) space representation of the actual positions that the body parts occupy with respect to the space of possibilities that a body part might occupy, e.g., the space into which a limb or a leg would reach; and
Allocentric (A) space representation of all of the space of possibilities that can be appreciated typically by visual, auditory or olfactory signals originating from the space beyond pericentric space and onto which a physical or knowledge interaction exchange can occur.

Accordingly, assuming existential and hedonic motivation, a motor plan generates a planned trajectory of cybernetic action. This action trajectory is then available for planning within the constraints of placement of the subject within the selected EPA space. Thus, a trajectory is planned in which:
a body part is moved in a kinematic trajectory that is the result of kinetic causal events;
the trajectory is set up as a series of choices that represent the alpha limit set (the inset of the stable limit point) with the omega limit set leaving the stable point;
the path length chosen from the trajectory connecting two limit points, the first being the trajectory required to move a body part from rest (the inset of the starting posture or the alpha limit set choice) and the trajectory chosen from the omega limit set into the next planned limit set point;
planning involves a motivational force field driving the placement of limit points in the EPA space; and
error correction involves the dynamical damping of trajectories to achieve a path of least action onto the omega limit with the possibility that a trajectory may not reach a stable limit point but rather be converted into a stable limit cycle (tremor) or a chaotic trajectory near the limit point.

With this background of human motor control, this disclosure provides a system and method for mathematically describing/modeling the movement system and, as an example, the movements associated with various diseases or conditions, including PD and parkinsonism.

The clinical diagnosis of idiopathic or Lewy-body PD is typically defined by the presence of four characteristics: bradykinesia, rest tremor, rigidity and postural instability. A clinical diagnosis of PD typically requires a patient to, at a minimum, exhibit bradykinesia plus either tremor or rigidity. See Postuma RB et al., MDS Clinical Diagnostic Criteria For Parkinson's Disease, 30 J. Mov. DISoRD. 12,1591-1601 (October 2015) (available online at https://www.ncbi.nlm.nih.gov/pubmed/26474316 #), incorporated by reference herein. While postural instability is a characteristic of PD, it typically does not appear until later in the course of the disease, and is not present in the early stages for purposes of diagnosis, An unequivocal improvement of the bradykinesia to dopaminergic treatment is supportive of the diagnosis of Lewy-body PD, as is the unilateral onset and persistent asymmetry in motor signs in the limb of onset. A. J. Hughes et at, Accuracy of Clinical Diagnosis of Idiopathic Parkinson's Disease: A Clinico-Pathological Study of 100 Cases , 55 J. NEUROL NEUROSURG PSYCHIATRY 3, 181 (1992) (available online at https://www.ncbi.nlm.nih.gov/pubmed/1564476), incorporated by reference herein.

Bradykinesia, a sign of PD, is clinically defined by the presence of the following features in mathematical terms:
1. Hypolinea, which is defined as a reduction in the amplitude or displacement of a body part xₑ when compared to the intended or planned movement xₚ ∴xₑ<xₚ
2. Hypokinesia, which is defined as a reduction in the velocity of the trajectory tracing the path of the

$${x}_{p} . ∴ \frac{\partial {x}_{e}}{\partial t} < \frac{\partial {x}_{p}}{\partial t}$$

3. Momentary Akinesia or Hesitations, which are complete but momentary arrests of motion along the path of planned movement, xₚ. These momentary arrests occur when

$$\frac{\partial {x}_{e}}{\partial t} = 0 \& 0 ms \leq t \leq 250 ms$$

4. Akinesia or Halts, which are complete and momentary arrests of motion along the path of planned movement, xₚ. These momentary arrests occur when

$$\frac{\partial {x}_{e}}{\partial t} = 0 \& t > 250 ms$$

5. Dyspalilinea, which is the failure in an iterated movement of period T to maintain the intended amplitude of the path x(t). A metric of this iterated function is the deviation (incremental increase) from the point of first return x(t₀) of the trajectory orbit as the iteration number increases.
   a. Normal palilinea is characterized by: x(t₀+T)=x(t₀)
   b. Dyspalilinea is characterized by: x(t₀+T)<x(t₀)
      See Robert Efron, The Minimum Duration of a Perception, 8 Neuropsychologia 1, 57-63 (1970), (available online at https://doi.org/10.1016/0028-3932(70)90025-4), incorporated herein by reference.

MDS-UPDRS is the general measure of the motor components of PD that are applied in research studies. The above mentioned features that characterize bradykinesia are assessed and then added together against the backdrop of a “normalized population,” Part III of the MDS-UPDRS is a summation of non-parametric ‘Z-scores’ given to each of 18 tasks. Of these, bradykinesia is the cardinal feature measured in parts 3.4, 3.5, 3.6, 3.7, 3.8 and 3.14 of the MDS-UPDRS.

In addition, gait (parts 3.10 and 3.11 of the MDS-UPDRS) is measured using the same features as bradykinesia (e.g. hypolinea, hypokinesia, momentary akinesia/hesitations, akinesia/halts, and dyspalilinea) described above. Gait measures are an assessment of bradykinesia and rigidity except that there is an additional translational motion taking place in an accelerating frame of reference (gravity) in which the subject is toppling forward and iteratively catching their accelerating center of gravity under a translating base of support.

Tremor is a periodic flexion-extension movement that can be considered as a stable limit cycle that is concentric around what should be a stable limit point defined as the endpoint of xₚ. Tremor is considered as a dynamical state caused by a driven paired oscillator that has missed the stable limit point and is instead trapped in a limit cycle. Under conditions that generate clinical dyskinesia, the trajectories become chaotic. Tremor in PD is measured in terms of its amplitude, its frequency, and its proportion of time in oscillation, divided by the time spent in the contextual state. There are three contextual states characterized in PD for tremor:
1. Resting tremor: a body part at rest relative to the gravitational field with no force exerted by the body to maintain the rest pose;
2. Postural tremor: the body part is at rest relative to the gravitational field with an antigravity force F=mg (m is body mass and g is the gravitational acceleration constant) needed to hold the part at rest pose; and
3. Action tremor: while in translation motion with a momentum imparted by the motor system, the trajectory of the intended action degrades because of discrete inability of the body part to move from one alpha limit set through the omega limit onto the omega limit set. Instead the body part occupies a cyclic limit set trajectory that leads to tremor around the trajectory.

Balance and posture are variances around the normal exploration of local limit space in which the ∇(xₚ−xₑ) necessary to keep the center of gravity within the boundaries of the base of support (BoS) are defined and bounded.

PD has been shown to be characterized by errors in motor planning in which the person with PD (PwPD) chooses too small a separation xₚ(a)—xₚ(b) between points a and b. These failures are likely errors in the interactions between Brodman 6 which is largely innervated by the D1 circuits (go circuits) in the cortico-thalamic loops. The failures of discrete trajectory motor planning lead to hypolineal motor movements. These are also manifested by the lack of normal agonist motor unit firing that causes rapid acceleration necessary to generate the torque appropriate to move a body part that normally is followed by corrective antagonist firing of the motor nerve. This leads to the rapid deceleration and small accelerations necessary to achieve landing on the stable limit point chosen for the planned action.

Systems and Methods to Assess a Patient State

Embodiments of this disclosure relate to a dynamical systems-based approach for quantifying and/or modeling a patient state in order to diagnose and/or measure the progress of a disease, such as PD. The availability of low-cost digital devices to quantify body movements makes it possible to develop an objective assessment system for motor system monitoring. Compared to more subjective approaches, a quantitative approach to medicine more accurately defines a patient's journey as a set of states in a trajectory across the life phases of growth, development, health, disease, and recovery. Disclosed herein is a mathematical framework that utilizes dynamical system modeling and phase plots to quantify the temporal properties of disease features, including, for example, bradykinesia associated with PD. The disclosed mathematical approach can significantly improve the ability to quantify drug effects in clinical trials in at least movement, neuromuscular, and multiple sclerosis disorders. Moreover, the disclosed framework ties the analysis methodology to the underlying human biology. The metrics will be directly related to established neurological features and neural circuits. While PD is discussed in this disclosure as the exemplary condition, the systems and methods of this disclosure are applicable to other diseases or patient states or conditions, including neuromuscular, neurodegenerative, or other physiological diseases, or other diseases of the cardiovascular, pulmonary, or digestive systems, among others.

Embodiments of this disclosure relate to systems and methods of measuring traits, signs, or symptoms characteristic of a disease or other abnormal patient state and determining or quantifying a level and/or a progression of the state. In examples, the system or method may include one or more of the following steps:
determine one or more traits, signs, or symptoms characteristic of a disease;
determine one or more measurements descriptive of the trait, sign, or symptom;
model those measurements, for example mathematically;
obtain the measurements descriptive of the trait, sign, or symptom from at least one subject that does not have the disease/state, for example a healthy individual, and use the model to transform the measurements into reference data;
obtain the measurements from another subject, e.g. a patient, and use the model to transform the measurements into patient data; and
compare the reference data to the patient data, to determine a level of the disease in the patient.

Determining traits, signs, or symptoms characteristic of a disease may include determining known, established traits, signs, or symptoms. For example, PD is a disease suitable for use in connection with methods and systems of this disclosure. Determining traits, signs, or symptoms of PD may include consulting any form of literature describing established traits, signs, or symptoms. Known traits, signs, or symptoms of PD include bradykinesia, tremors, imbalances, and gait abnormalities, as discussed above. See Christopher G. Goetz et al., Movement Disorder Society-sponsored revision of the Unified Parkinson's Disease Rating Scale (MDS-UPDRS): Scale Presentation and Clinimetric Testing Results, 23 MOV. DISORD. 15, 2129-2170 (Nov. 15, 2008) (available online at https://www.ncbi.nlm.nih.gov/pubmed/19025984), incorporated by reference herein. For less researched diseases or patient states, or diseases/states otherwise having less established traits, signs, or symptoms, this step may include identifying the traits, signs, or symptoms characteristic of the disease/state by observing patients with the disease or performing tests to identify such traits, signs, or symptoms. Thus, traits, signs, or symptoms characteristic of a disease may be already known or newly-identified.

Another step in the method and system may include determining one or more measurements descriptive of the trait, sign, or symptom. Such measurement is not particularly limited, and may include cognitive and/or motor-related measurements. For example, referring again to PD, one such sign is bradykinesia, which is slowness of movement as described above. Useful measurements descriptive of bradykinesia include displacement, velocity, and acceleration of movements.

Another step in the method and system may include modeling the measurements descriptive of the trait, sign, or symptom. This modeling can be mathematical modeling, as described above. For example, displacement, velocity, and acceleration of movements descriptive of bradykinesia can be mathematically modeled.

Another step in the method and system may include obtaining measurements descriptive of the trait, sign, or symptom from at least one subject that does not have the disease, for example a healthy individual. Using PD and bradykinesia as the example, measurements of a healthy individual may be made using any suitable measurement device or system. Low-cost digital devices to quantify movements may be used to develop an objective assessment system for motor system monitoring.

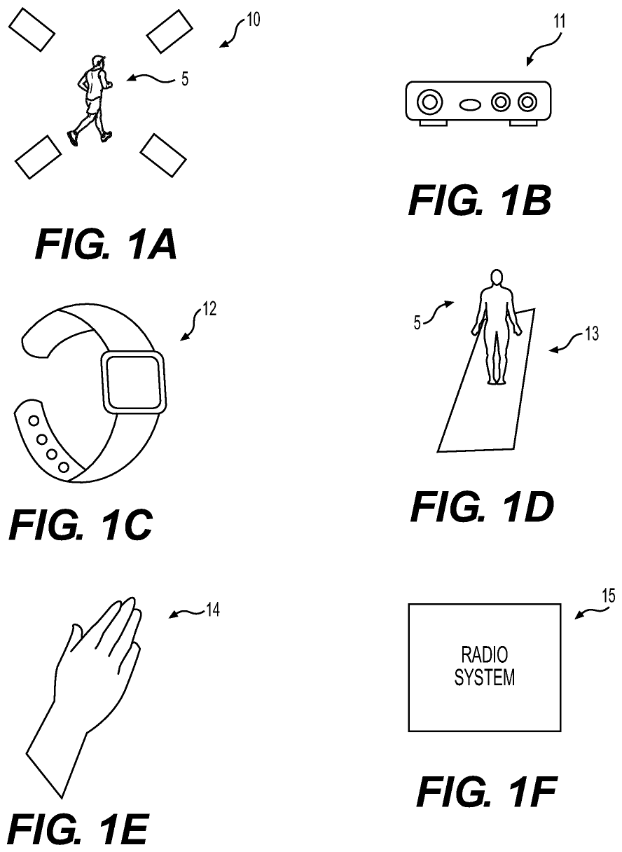

Referring to FIGS. 1A-1F, such exemplary systems or devices configured for motion capturing are shown (this includes voice and speech production as examples of vocal movement). These systems or devices can be divided into categories as shown in FIGS. 1A-1F. For example, FIG. 1A illustrates an optical system 10 monitoring the motion of a user 5. Optical system 10 is not particularly limited, and may be a tag or tagless optical system configured to monitor any voluntary/involuntary movements from user 5. FIG. 1B illustrates an image processing system 11, which may include any number of suitable functions, e.g., pose analysis. FIG. 1C illustrates an example of a wearable device 12, a watch. Wearable device 12 may include any number of sensors (not shown) at suitable locations for measuring the inertial system of a wearer. Wearable device 12 may be worn on any portion of the body, and its shape or configuration is not particularly limited to the example shown. FIG. 1D illustrates a floored system 13, which may include any number of floor sensors configured to measure metrics associated with user 5 standing and/or moving along said floor sensors. Said floor sensors may be of any suitable size, shape, or form, e.g., an instrumented mat or force plate. FIG. 1E illustrates an example of a contact base system 14 (electrical or resistive), for example a glove, which may be in the form of a wearable, but not limited thereto. FIG. 1F illustrates a radio frequency-based system 15, which is not particularly limited and may be an active or passive system. Thus, systems/devices 10-15, shown in FIGS. 1A-1F, can include at least one of wearable sensors, inertial sensors, accelerometers, cameras, electromyography systems, strain gauges, motions trackers, force plates, etc. Furthermore, any one of systems/devices 10-15 shown in FIGS. 1A-1F may be used alone or in combination with other systems/devices. For example, user 5 in optical system 10 may also be wearing wearable device 12.

These exemplary digital systems/devices shown in FIGS. 1A-1F have the potential to not only enable more accurate disease quantification, but also offer consistency of data for longitudinal studies, accurate stratification of patients for entry into trials, and the possibility of automated data capture for remote follow-up. The devices mentioned above are exemplary, and other devices for measuring a patient state may be contemplated. The methods disclosed herein are independent of any specific motion capture system and can be generalized to any device or disease or patient state.

Measurements descriptive of the trait, sign, or symptom may be obtained from a healthy, reference subject. Continuing to use PD and bradykinesia as the example, measurements may be obtained as the healthy subject performs a motion, such as a pronation-supination task with a hand. Motion metrics, including displacement, velocity, and acceleration, can be measured over time. These metrics can be obtained for a number of healthy individuals to generate population-level statistics. The metrics can then be normalized and converted to a “score”, e.g., z-score, by subtracting the mean (μ) and dividing by the standard deviation (σ) of the metric in the healthy population:

$${z}_{metric} = \frac{metric - {μ}_{metric ( healthy )}}{{σ}_{metric ( healthy )}}$$

Using the model, the reference data obtained may then be plotted, for example, in a three-dimensional, dynamic displacement, velocity, and acceleration graph.

Another step in the method and system may include obtaining the measurements from another subject, e.g. a patient that may be symptomatic of the disease or other abnormal condition. Such measurements can be obtained in the same or different fashion as described above for the healthy subject. Using the mathematical model, the measurements are transformed into patient data that may be graphed and displayed in a same or similar manner as described above. Demographics also may be obtained for that patient. In some embodiments, measurements are made for a number of healthy individuals, including individuals of different demographics. The different demographics may include age, gender, etc.

Another step in the method and system may include comparing the data from the patient to the reference data from the healthy individual, to determine a level of the disease in the patient. In some examples, the comparison can be made between a patient and a healthy subject that share demographics. The data comparison may be characterized by a score for the particular trait, sign, or symptom. In some embodiments, the data is normalized by age, and in some cases, gender. It is further noted that data comparison is not limited to comparing reference data from a healthy population to data from a patient. Measurements of a patient can be taken periodically, in intervals, e.g., daily, weekly, annually, etc., for a duration of time, and comparison between the patient's data sets from different times/dates can be made. This may provide a mapping of the progression of traits, signs, or symptoms associated with the disease.

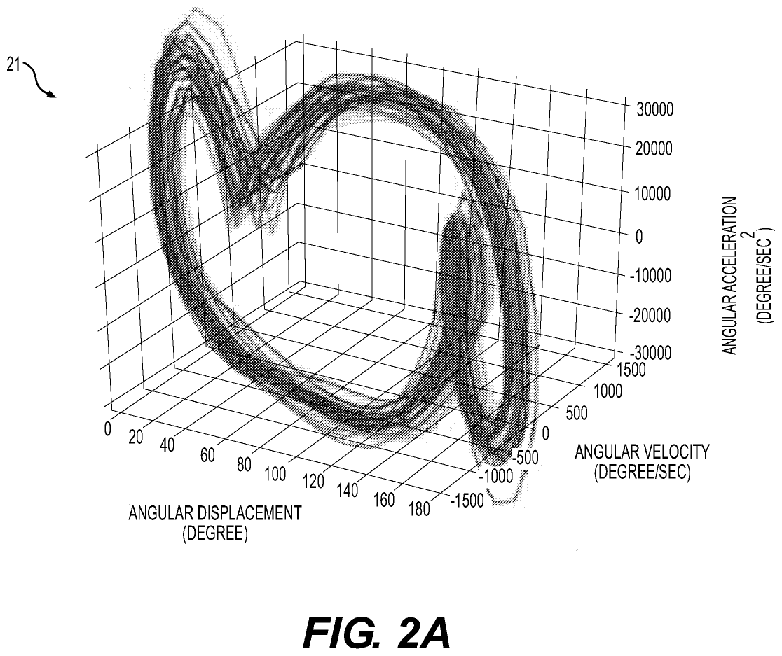

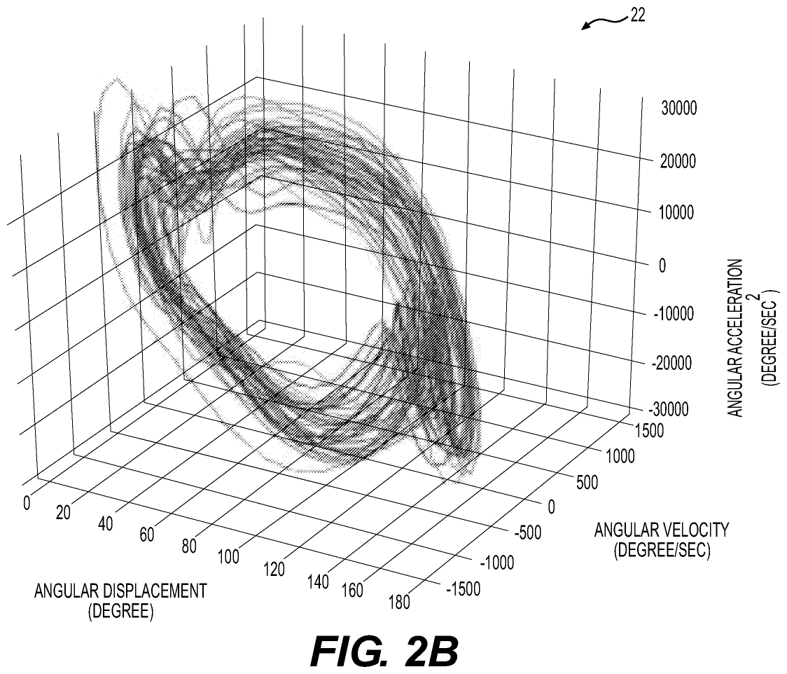

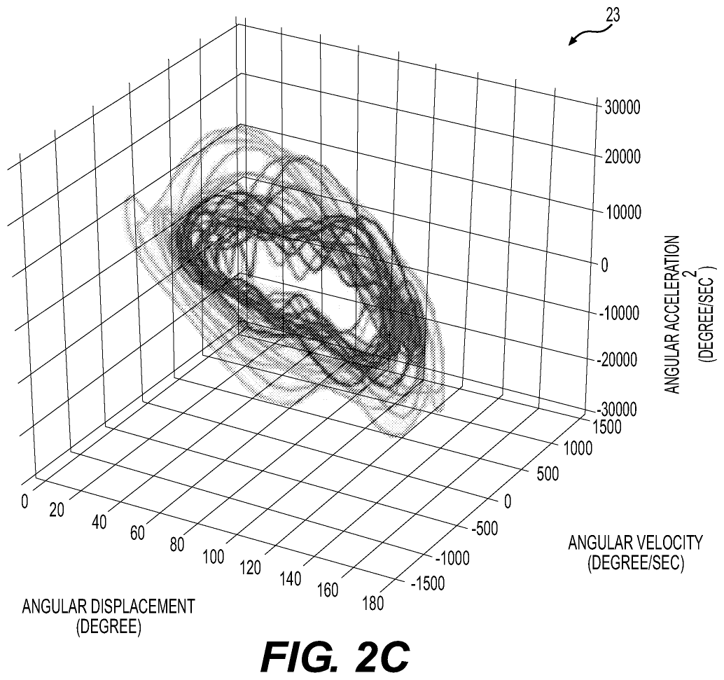

Referring to FIGS. 2A-2C, 3A-3H, 4A-4C, and 5A-5F, an exemplary process of the steps discussed above is shown via a series of graphs and plots. To obtain the data presented in FIGS. 2A-2C, 3A-3H, 4A-4C, and 5A-5F, the following study was conducted. 129 PD participants were assessed during the baseline visit of an ongoing phase 2 clinical trial, SPARK (NCT03315523, digital subset) using a wearable Inertial Measurement Unit (IMU) sensor-based movement monitoring system. (See Mancini M, King L, Salarian A, Holmstrom L, McNames J, Horak FR Mobility Lab to Assess Balance and Gait with Synchronized Body-worn Sensors. J Bioeng Biomed Sci. 2011; Suppl 1:007. doi:10.4172/2155-9538.S1-007), incorporated by reference herein. Data was acquired from five synchronized sensors (L5 lumbar, bilateral wrists, and top of each foot) while the participants completed a series of activities derived from MDS-UPDRS part III, including repetitive arm and leg movements, walking, sitting, and standing still, referred to as the Quantitative Movement Assessment for PD (QMA-PD). Comparison data were obtained from 24 healthy volunteers also completing the QMA-PD, under the same protocols. A visualization system was developed to summarize patient state using phase plots. These plots are derived from the sensor time series data and use a point trajectory in phase space to represent changes in the task dynamics over time as the participants perform the different activities. Signal features were extracted from the phase plane chosen to reflect phenomena of motion that comprise the clinical definition of PD motor signs (e.g. bradykinesia and tremor) and traditionally make up the scoring guidelines for observation during items on MDS-UPDRS part III. Digital bradykinesia and tremor scores were calculated by normalizing these features based on distributions of the metrics from the healthy volunteers in the study. Phase plots (shown in FIGS. 2A-2C, 3A-3H, 4A-4C, and 5A-5F) were derived based on said calculations.

Plots 21, 22, 23 shown in FIGS. 2A-2C, respectively, are dynamical analysis revealing the progression of bradykinesia. Specifically, plots 21, 22, 23 compare dynamics of the wrist pronation-supination repetitive task in healthy volunteers, i.e., MDS-UPDRS score 0 in plot 21, to PD participants at different stages of disease severity, i.e., MDS-UPDRS score 15 in plot 22 and MDS-UDPRS score 52 in plot 23.

As shown in plot 21, healthy volunteers at the same age, with a MDS-UPDRS of 0, may share a normal distribution of angular displacement, velocity, and angular acceleration of various limbs with respect to time. Similarly, as shown in plot 23, patients with PD with clearly visible bradykinesia symptoms, for example, having a MDS-UPDRS score of 52, may have significantly reduced angular displacement, velocity, and acceleration of limbs with respect to time as compared to healthy subjects. Similar data was obtained from such patients at various intermediate levels, e.g., MDS-UDPRS score 15 shown in plot 22.

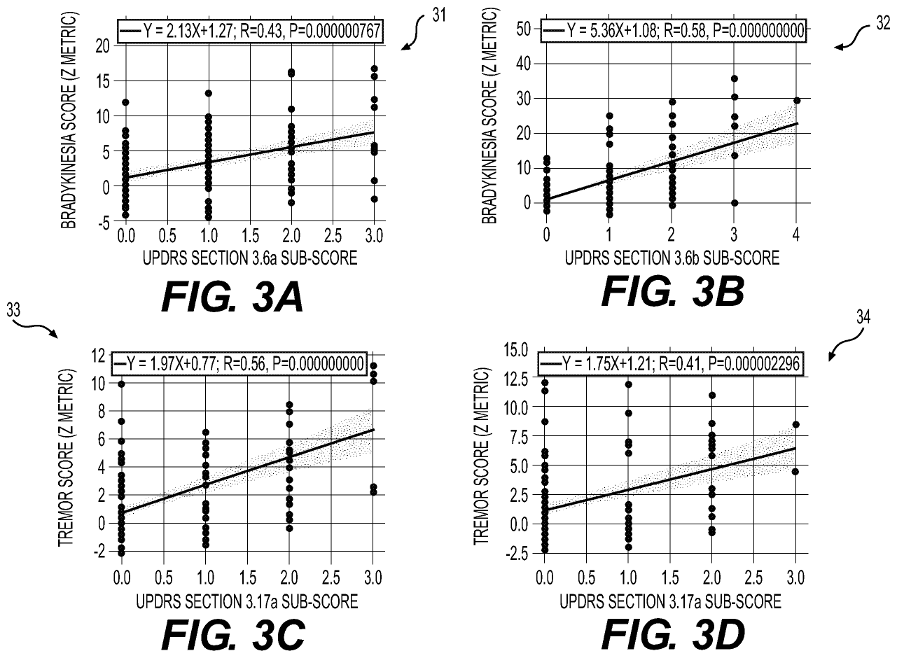

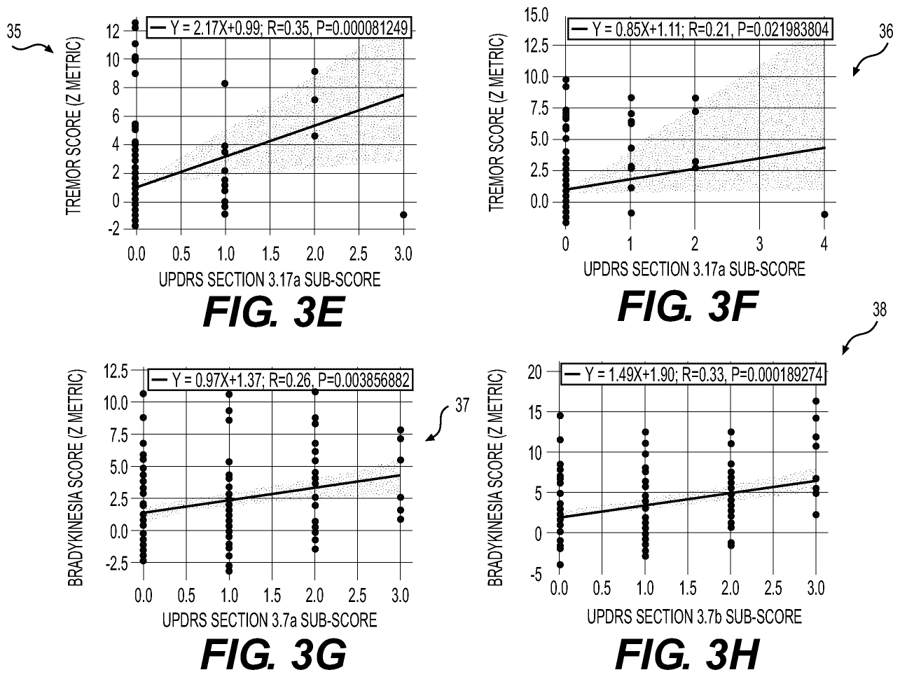

Plots 31-38 of FIGS. 3A-3H illustrate digital bradykinesia and tremor scores calculated for the different limbs. Specifically, plots 31, 33 involve measurements of the right wrist, plots 32, 34 involve measurements of the left wrist, plots 35, 37 involve measurements of the right foot, and plots 36, 38 involve measurements of the left foot. Plots 31-38 show a correlation between the digital bradykinesia and tremor scores calculated as a summation of the metric scores described in paragraph and the MDS-UPDRS part III sub-scores provided to patients. For example, as shown in FIGS. 3A-3H, the points represent the digital bradykinesia and tremor scores for the individual PD participants and the line represents the linear fit modeling their relationship with the corresponding MDS-UPDRS part III sub-scores. The R values represent the correlation coefficients and P values capture the statistical significance of the linear relationship.

Moreover, plots 31-38 illustrate differences in bradykinesia and tremor scores between patients provided with identical MDS-UPDRS part III sub-scores. Such differences highlight possible advantages of the above-discussed mathematical approaches in identifying differences between signs, symptoms, or traits, which may not be captured by standard qualitative assessments, e.g., MDS-UPDRS. For example, as shown in FIGS. 3A, patients given the same MDS-UPDRS Section 3.6a sub-score of 3.0 did not have the same digital bradykinesia score, when assessed mathematically. Rather, some patients had lower digital bradykinesia scores (less than 5) and some patients had higher digital bradykinesia scores (greater than 5), indicating some level of variance not captured by qualitative assessments, e.g., MDS-UPDRS.

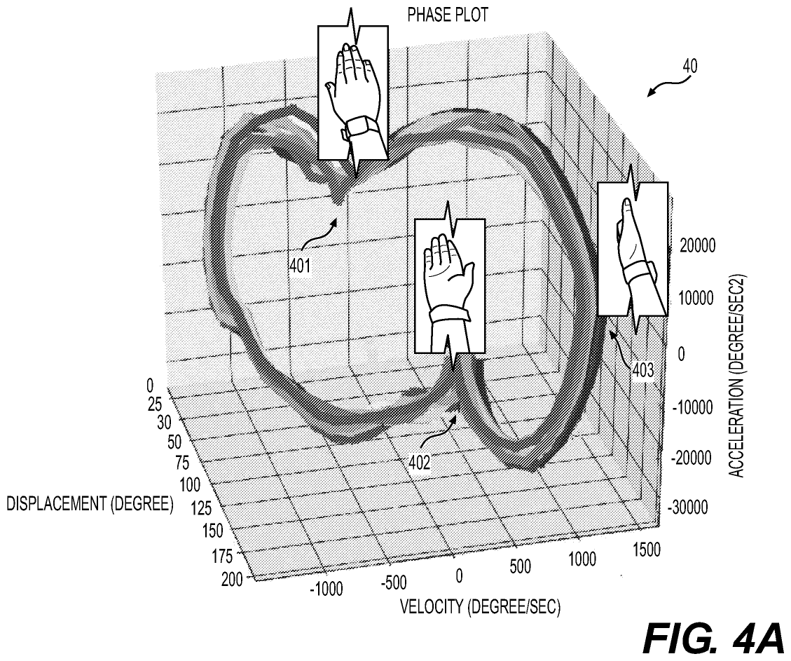

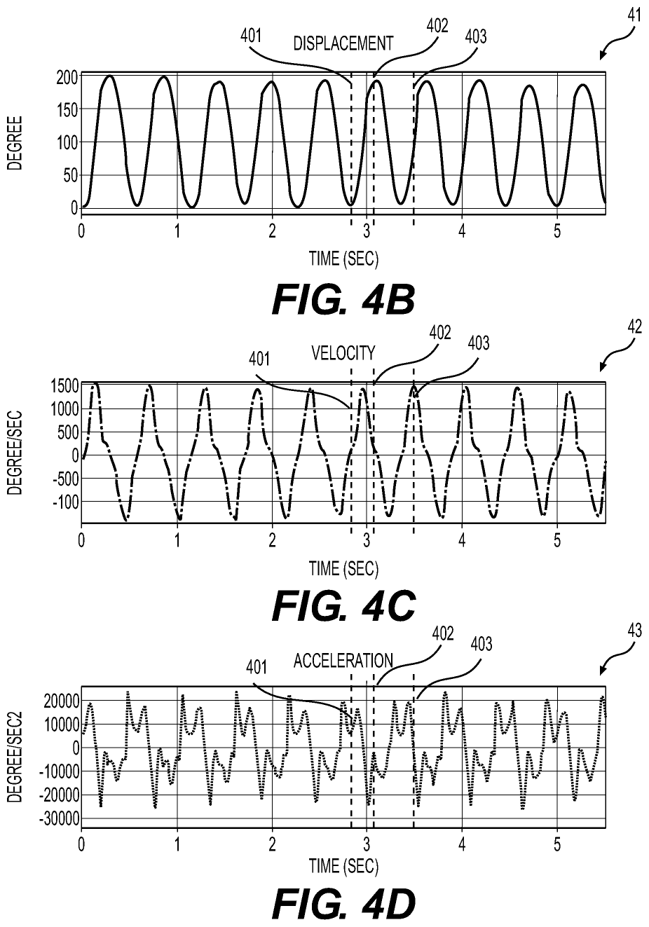

In some embodiments, phase plots can be used to represent all possible states of a dynamical system and show a relationship between various states (e.g. position, velocity and acceleration) as they evolve over time. Such plots are shown in plots 40-43 of FIGS. 4A-4D.

Phase plot 40 of FIG. 4A illustrates the relationship between the angular acceleration, velocity, and displacement of the wrist sensor as subjects perform the pronation-supination task in the QMA. Points 401, 402, and 403 correspond to different points of the plot during the transition between various positions of subjects' wrists (as shown) during the pronation-supination task. The rotation of the patient's hand is thus converted into patient or reference data that can then be compared to other patient and/or reference data to determine the state of a healthy person and/or a patient. Plot 41 of FIG. 4B illustrates the displacement of subjects' wrist while performing pronation-supination, plot 42 of FIG. 4C illustrates the velocity of subjects' wrist while performing pronation-supination, and plot 43 of FIG. 4D illustrates the acceleration of subjects' wrist while performing pronation-supination.

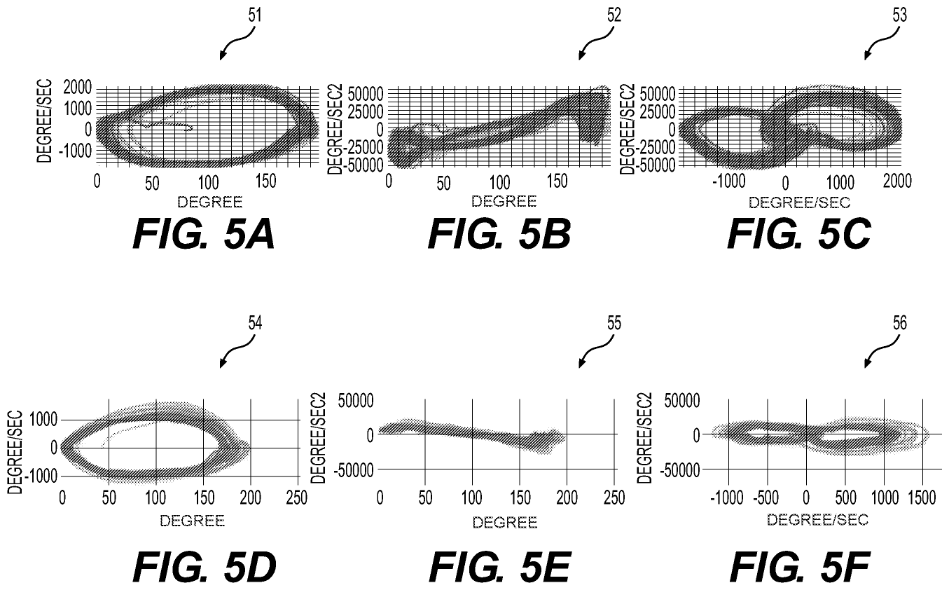

Another example of the application of the disclosed dynamical system modeling methods is demonstrated in FIGS. 5A-5F. Plots 51-56 show an exemplary dynamical analysis of a healthy volunteer's forearm and a PD patient's forearm showing bradykinesia metrics. When a patient performs a particular movement, for example, rotating their wrist or forearm, a device may measure angular displacement, velocity, and acceleration. Plots 51, 52, 53 show exemplary typical 2D phase plots capturing angular velocity vs. angular displacement (5A), angular acceleration vs, angular displacement (SB), and angular acceleration vs. angular velocity (5C), respectively from left to right, for a healthy volunteer. Meanwhile, plots 54, 55, 56 show exemplary typical 2D phase plots capturing angular velocity vs. angular displacement (5D), angular acceleration vs. angular displacement (5E), and angular acceleration vs. angular velocity (5F), left to right respectively for a patient with PD. Thus, mathematical approaches in measuring metrics of symptoms, e.g., displacement, velocity, acceleration, may assist in identifying individuals with diseases by comparing the plotted metrics to reference plots from healthy volunteers.

In addition to the metrics discussed above, it is noted that a plurality of metrics may be selected to quantify the kinematics of PD: bradykinesia in each arm and leg, resting tremor in each arm, gait disturbance, tremor with held posture, and postural instability based on data obtained from inertial sensors. Such metrics reflect phenomena of movement derived from the clinical definition of PD motor signs and traditionally make up the scoring guidelines for observation during items on MDS-UPDRS part III Metrics include:
Left wrist/right wrist
   Wrist rotations bradykinesia composite (Z-score sum)
      1. Angular displacement median (degree)
      2. Angular velocity median (degree/sec)
      3. Angular velocity repetitions per second (rps) non-parametric coefficient of variation (npcv) (unitless)
      4. Angular displacement npcv (unitless)
      5. Angular velocity npcv (unitless)
   Resting Tremor (Z-score)
      1. Angular velocity spectral entropy (unitless)
Left leg/right leg
   Leg lifts bradykinesia composite (Z-score sum)
      1. Linear displacement median (cm)
      2. Linear velocity median (cm/sec)
      3. Linear velocity rps npcv (unitless)
      4. Linear displacement npcv (unitless)
      5. Linear velocity npcv (unitless)
Left foot/right foot
   Toe taps bradykinesia composite (Z-score sum)
      1. Angular displacement median (degree)
      2. Angular velocity median (degree/sec)
      3. Angular velocity rps npcv (unitless)
      4. Angular displacement npcv (unitless)
      5. Angular velocity npcv (unitless)
   Resting Tremor (Z-score)
      1. Angular velocity spectral entropy (unitless)
   Walk bradykinesia composite (Z-score sum)
      1. Angular displacement median (degree)
      2. Angular velocity median (degree/sec)
      3. Angular velocity rps npcv (unitless)
      4. Angular displacement npcv (unitless)
      5. Angular velocity npcv (unitless)
   Turn bradykinesia composite (Z-score sum)
      1. Max turn velocity (degree/sec)
      2. Inter-quartile range (IQR) turn velocity (degree/sec)
Lumbar
   Postural Sway (Z-score)
      1. Linear acceleration spectral entropy (unitless)
   Resting Tremor (Z-score)
      1. Linear acceleration spectral entropy (unitless)
         It is noted that the Power Spectral Density from linear time series can be computed using the Welch method with a Harming window as implemented in SciPy. (See Welch P. The use of fast Fourier transform for the estimation of power spectra: A method based on time averaging over short, modified periodograms. IEEE Transactions on Audio and Electroacoustics. 1967; 15 (2) 70-73. doi: 101109/TAU.0.1967.1161901; see also Virtanen P, Gommers R, Oliphant TF, et al. SciPy 1.0: fundamental algorithms for scientific computing in Python. Nature Methods. 2020; 17(3):261-272. doi: 10.1038/s41592-019-0686-2), incorporated by reference herein. The entropy quantifying non-uniformity of the power spectra may be computed as follows:

$$Spectral Entropy = - {\sum }_{i = 1}^{N} {p}_{i} ln ( {p}_{i} )$$

(See Gramfort A, Luessi M, Larson E, et al. MEG and EEG data analysis with MNE-Python. Front Neurosci. 2013; 7:267.doi:10.3389/fnins.2013.00267), incorporated by reference herein. Moreover, npcv from linear time series may be computed as npcv=MADM/median, where MADM is Median Absolute Deviation.

Additional details of an exemplary embodiment of measuring and modeling of a pronation-supination task will now be described. An inertial measurement unit on the wrist can be used to instrumentize the pronation and supination task. The 3D orientation (θᵣₒₗₗ, θ_{yaw}, θ_{pitch}) of the wrist can be estimated by integrating gyroscopes and fusing this data with accelerometer and magnetometer data using Madgwick implementation of the AHRS (Attitude and Heading Reference System) algorithm. Madgwick algorithm compensates drift from the gyroscopes integration by reference vectors, namely gravity (from accelerometer), and the earth magnetic field (from magnetometer). For example, data from IMU (inertial measurement unit) may first be subjected to a high pass filter of 0.5 Hz to remove drift and gravity components from the acceleration. To be invariant of IMU (inertial measurement unit) frame of reference, orientation data can be rotated to the first PCA (principal component analysis) component using singular value decomposition and finally various state parameters can be computed using differential and integral transforms. See Oppenheim AV, Schafer RW, Buck JR Discrete-Time Signal Processing (2nd Ed.). Prentice-Hall, Inc.; 1999, incorporated by, reference herein. Mathematically:

Given a 6-dimensional time series from IMU representing angular velocity and linear acceleration; D=[d₁, . . . , dᵣ]∈ [symbol] ^{6×T}, where dₜ=[\dot{θ}ᵣₒₗₗ, \dot{θ}_{yaw}, \dot{θ}_{pitch}, aₓ, a_{y}, a_{z}] and two orthogonal transformation vector p_{θ}ε [symbol] ^{1×3} for angular velocity and Pα∈ [symbol] ^{1×3} for linear acceleration, which maximizes data variance captured by the low-dimensional projection \dot{θ}∈ [symbol] ^{1×T} and α ∈ [symbol] ^{1×T}, such as:

\dot{θ}(t)=pD_{\dot{θ}},

α(t)=pD_{α}

where p minimizes the [symbol] ₂-norm reconstruction error between the projected data points with the original 3D angular velocity or linear acceleration:

[symbol]

ₓ(p)=min∥X−p^{T}pD∥_{F}²

The angular position and acceleration respectively can be computed from angular velocity (\dot{θ}) as follows:

$$θ ( t ) = \int \dot{θ} dt ,$$

$$\ddot{θ} ( t ) = \frac{{d}^{2} \dot{θ}}{{dt}^{2}}$$

The linear velocity and position respectively can be computed from linear acceleration (a) as follows:

v(t)=∫αdt,

x(t)=∫∫αdt²

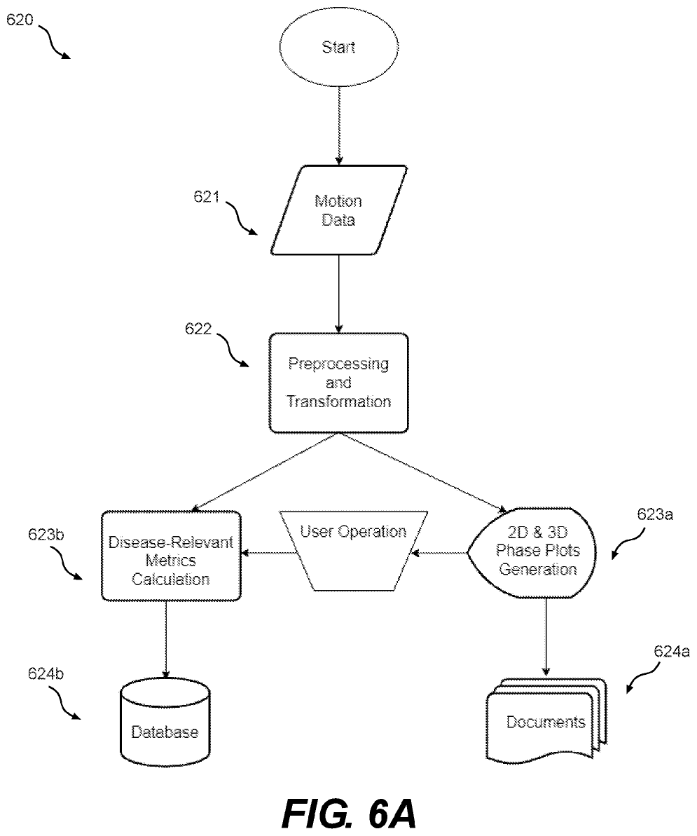

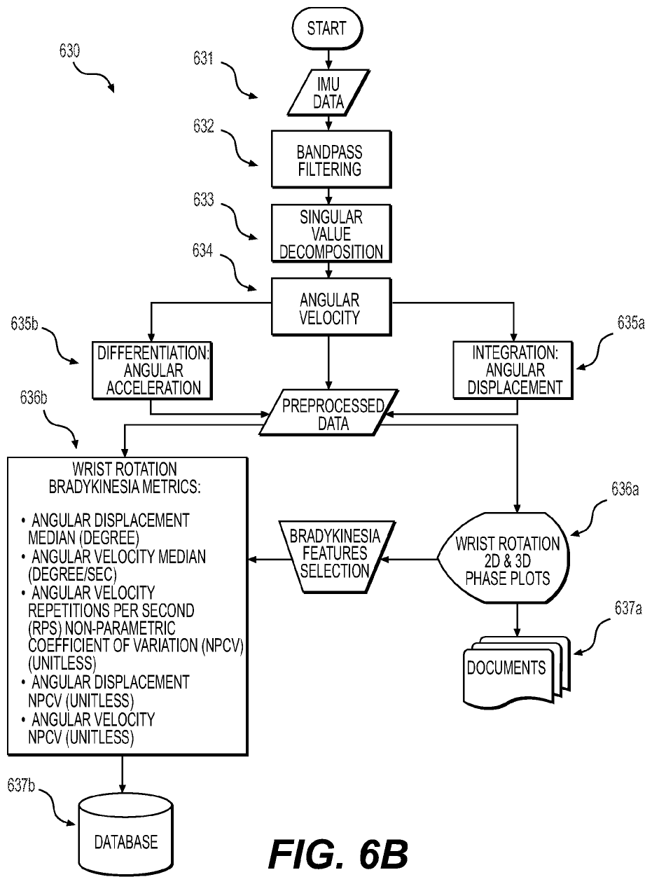

Referring to FIG. 6A, a process 620 for preparing disease-relevant metric calculations, such as those discussed above, is shown. Process 620 may include a first step 621 of collecting any necessary data, e.g., motion data, from the subject. In a second step 622, the collected data may be preprocessed and transformed before being used for phase plot generation, i.e., step 623a, and disease-relevant metrics calculation, i.e., step 623b. It is noted that the generated two-dimensional and three-dimensional phase plots from step 623a may be examined and used to inform disease-relevant metrics calculations of step 623h. In step 624a, the generated phase plots may be stored as documents, and in step 621b, the disease-relevant metrics may eventually be output into a database.

Referring to FIG. 613, a process 630 more specific to the context of bradykinesia metric calculations is shown. Process 630 illustrates the workflow for forearm bradykinesia metrics calculation in the context of pronation-supination task. In step 631, IMU (inertial measurement unit) data from a wrist sensor is first collected from the subject. The collected data may be preprocessed via bandpass filtering (step 632) and singular value decomposition (step 633), or any other suitable preprocessing steps. Based on the preprocessed data, a metric, angular velocity, may be identified in step 634, and then transformed via a step 635a of integration, and a step 635b of differentiation. Parameters resulting from integration step 635a, e.g., angular acceleration, and differentiation step 635b, e.g., angular displacement, may be used for wrist rotation phase plot generation (step 636a) and bradykinesia metrics calculation (step 636b). It is noted that the generated two-dimensional and three-dimensional phase plots from the pronation-supination task can be examined and used to inform bradykinesia metrics generation. Said metrics may include, for example, angular displacement median, angular velocity median, angular velocity repetitions per second non-parametric coefficient of variation (npcv), angular displacement npcv, and angular velocity npcv. The phase plots may be stored as documents in a step 637a, and bradykinesia metrics from the pronation-supination task are eventually output into a database in a step 637b.

Instead of relying on the subjective MDS-UPDRS measurements of a particular physician, a patient's angular displacement, velocity, and acceleration may be measured and compared to prior, reference data in an objective and mathematical manner. This offers a one-to-one mapping to the corresponding metrics, thereby allowing a direct comparison between patients' data sets and reference data. The detection of even small reductions in angular displacement, velocity, and acceleration over time is possible, and a graphical analysis may indicate the onset of PD before a doctor observes any difference in a patient. Further, the progress of PD can be more accurately measured for a patient with PD, as the disclosed methods herein may measure and detect any change in displacement, velocity, or acceleration. Additional benefits include a reduction in cost and time to complete clinical trials, including the potential for patients to be screened at home for diseases without having to go in to a physical clinical facility.

The mathematical treatment of the bradykinesia may be presented in the context of the pronation and supination task; however, this can be expanded to other tasks/motions, including repetitive tasks/motions, without loss of generality. Pronation and supination typically require a person to flip their palm either face up or face down. In Table I below, the MDS-UPDRS scoring criteria for the pronation and supination is described (MDS-UPDRS. official Working Document).

TABLE 1
The MDS-UPDRS scoring criteria for the pronation and supination

| | Rhythm | | Amplitude |
|---|---|---|---|
| Score | (Interruptions) | Speed | Decrement |
| 0 | No interruptions | Normal speed | No decrement |
| 1 | 1 to 2 | Slightly slowing | Near end of sequence |
| 2 | 3 to 5 | Mild slowing | Midway in sequence |
| 3 | >5 | Moderate slowing | Starting after first sequence |

| | |
|---|---|
| 4 | Cannot or can only barely perform the task |

The method described herein may be repeated for a plurality of traits, signs, or symptoms for a disease or other condition. This may result in a number of scores, each characterizing a corresponding trait, sign, or symptom. As an example, those scores can be combined and weighted to produce an overall score for a disease state. Such an overall score can correspond to scores of conventional methods, so that commonly used scoring systems may be maintained. For example, for PD, the process disclosed herein may be used to determine scores for dyskinesia, tremor, gait, balance, etc., and then combined to result in a score understood according to the MDS-U PDRS scale used for PD. Goetz et al., supra 2129-2170.

The methods and systems of this disclosure may be used to measure the effects of a proposed, novel therapeutic/drug on the progression of a disease, for example PD. The methods and systems provide more reliable and sensitive clinical outcomes assessments, as compared to conventional clinical rating scales, such as the MDS-UPDRS scale used for PD. Goetz et al, supra 2129-2170. Previously, a neurologist or physician would observe a patient's movement, assess that movement based on the physician's knowledge/experience of prior cases in a subjective fashion, and then provide a subject score based on that observation/assessment. The disclosed methods and systems quantify a patient's movements and store that movement in a data format. That data may then be compared to prior data of healthy and/or diagnosed patients, in order to determine whether the patient has the condition. As an example, motion capture technology may detect variations in velocity of a person's limb movement that may not be readily visible to a physician. Accordingly, PD onset can be detected earlier, and changes in PD progress can be documented. Thus, the disclosed methods allow for a sensitive machine to diagnose and monitor the progress of PD and other diseases quickly. The methods and systems also provide better disease diagnosis, and a better understanding of disease progression/regression. For example, embodiments of this disclosure may be used in clinical trials for a proposed therapeutic. Benefits of such use, as compared to conventional clinical methods, include shorter, more efficient, and less expensive clinical trials, and a reduction in the number of patients and patient visits needed to conduct the trial.

A computer that may be configured to execute techniques described herein, according to exemplary embodiments of the present disclosure. Specifically, the computer “platform” as it may not be a single physical computer infrastructure) may include a data communication interface for packet data communication. The platform may also include a central processing unit (“CPU”), in the form of one or more processors, for executing program instructions. The platform may include an internal communication bus, and the platform may also include a program storage and/or a data storage for various data files to be processed and/or communicated by the platform such as ROM and RAM, although the system may receive programming and data via network communications. The system also may include input and output ports to connect with input and output devices such as keyboards, mice, touchscreens, monitors, displays, sensors, etc. The system also may be configured to connect exemplary systems or devices configured for monitoring subjects, including those shown in FIGS. 1A-1F, Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a general or special purpose computer and/or data processor that is specifically programmed, configured, and/or constructed to perform one or more computer-executable instructions for implementing the disclosed methods. While aspects of the present disclosure, such as certain functions, may be described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), Cloud Computing, and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. AH or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

It will be apparent to those skilled in the art that various modifications and variations can be made to the disclosed systems and methods without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A medical process, comprising:
identifying one or more symptoms;
determining at least one measurement descriptive of the one or more symptoms;
creating a model of the at least one measurement;
obtaining the at least one measurement descriptive of the one or more symptoms from at least one symptomless subject;
using the model to transform the at least one measurement from the at least one symptomless subject into a reference data;
obtaining the at least one measurement descriptive of the one or more symptoms from at least one subject with the one or more symptoms;
using the model to transform the at least one measurement from the at least one subject with the one or more symptoms into a patient data; and
comparing the reference data to the patient data.

2. The medical process of claim 1, wherein the at least one measurement includes cognitive or physical movement related metrics.

3. The medical process of claim 1, wherein creating the model of the at least one measurement includes creating a mathematical model.

4. The medical process of claim 1, further comprising converting the at least one measurement from the at least one symptomless subject into a reference score, and converting the at least one measurement from the at least one subject with the one or more symptoms into a patient score.

5. The medical process of claim 1, further comprising mapping the reference data to create a reference graph, and mapping the patient data to create a patient graph.

6. The medical process of claim 1, wherein the at least one measurement descriptive of the one or more symptoms from at least one subject with the one or more symptoms is obtained in time intervals.

7. The medical process of claim 1, wherein the one or more symptoms are attributed to a disease including Parkinson's Disease.

8. The medical process of claim 1, wherein the one or more symptoms include bradykinesia, gait, and/or tremor.

9. The medical process of claim 1, wherein the at least one measurement includes movement related metrics including displacement, velocity, and acceleration of movements.

10. The medical process of claim 1, wherein obtaining the at least one measurement descriptive of the one or more symptoms from the at least one subject with the one or more symptoms includes monitoring the at least one subject with the one or more symptoms while completing at least one task.

11. The medical process of claim 10, wherein the at least one task includes movement-related tasks.

12. The medical process of claim 10, wherein the at least one task includes wrist rotation, leg lifts, toe taps, walking, and/or postural sway.

13. The medical process of claim 1, wherein the at least one measurement is obtained via a subject monitoring system.

14. The medical process of claim 13, wherein the subject monitoring system is configured to monitor a motion of the at least one symptomless subject and the at least one subject with the one or more symptoms.

15. The medical process of claim 13, wherein the subject monitoring system includes at least one sensor configured to obtain the at least one measurement.
